(19) United States
(12) Patent Application Publication
(10) Pub. No.: US 2010/0003265 A1
(43) Pub. Date: Jan. 7, 2010

(54) Isolation, expansion and uses of tumor stem cells

(75) Inventors: Bjorn Scheffler, Remagen-Oberwinter (DE); Antje K. Goetz, Remagen-Oberwinter (DE); Dennis A. Steindler, Gainesville, FL (US)

Correspondence Address: Edwards Angell Palmer & Dodge LLP, P.O. Box 55874, Boston, MA, 02205, US

(73) Assignee: University of Florida Research Foundation, Gainesville, FL (US)

(21) Appl. No.: 12/381,219

(22) Filed: Mar. 9, 2009

Related U.S. Application Data
(63) Continuation of application No. PCT/US07/19806, filed on Sep. 11, 2007.
(60) Provisional application No. 60/843,660, filed on Sep. 11, 2006.

(51) Int. Cl. A61K 39/395; C12N 5/06; G01N 33/574; A61P 35/00

(52) U.S. Cl. 424/174.1; 435/325; 435/7.23

(57) ABSTRACT

Disclosed are methods for isolating cell populations enriched in tumor stem cells (cancer stem cells), and isolated cell populations substantially enriched in cancer stem cells that are tumorigenic in vivo. Also provided are new methods of tumor diagnosis and classification and personalized methods of treatment for subjects with tumors, based on the availability of populations of cancer stem cells derived from the subject's tumor using the disclosed methods.

### RELATED APPLICATIONS

This application is a continuation of international application PCT/US2007/019806 filed Sep. 11, 2007, which claims the benefit of U.S. Provisional Application 60/843,660 filed Sep. 11, 2006. The entire contents of each of the aforementioned applications are hereby incorporated herein by reference.

### STATEMENT OF U.S. GOVERNMENT INTEREST

Funding for the present invention was provided in part by the Government of the United States under Grant Nos.: NIH/NINDS NS37556 and 5R21NS46384-2 from the National Institutes of Health. Accordingly, the Government of the United States has certain rights in and to the invention.

### FIELD OF THE INVENTION

The invention generally relates to cellular compositions and methods of production thereof, useful for the diagnosis and treatment of cancer. More specifically, the invention relates to methods of isolating cancer stem cells and non-carcinogenic cells from tumors and preparing enriched preparations thereof.

### BACKGROUND

Despite decades of research, brain tumors and other neurological disorders continue to cause high rates of morbidity. Brain tumors in particular show increased mortality when highly migratory active and proliferative tumor cells become, or already are, resistant to radio- and chemotherapy (Merchant and Fouladi, 2005; Henson, 2006; Massimino and Biassoni, 2006). Recently it has been proposed that cancer stem cells (CSC) may represent the driving force behind some of the deadliest entities among brain tumors, e.g., glioblastoma multiforme (GBM) and anaplastic ependymoma (AEp) (Polyak and Hahn, 2005; Sanai et al., 2005; Taylor et al., 2005).

Diagnosis, treatment and prognosis of most human tumors of the central nervous system (CNS) is presently based almost exclusively on histopathological criteria such as cytological appearance, necrosis, and tumor cell or endothelial cell proliferation. There is a fundamental lack of knowledge about the cells of origin of primary neoplasias of the CNS. This basic lack of knowledge has hindered efforts to develop effective therapies for these tumors.

Despite the interest in cancer stem cells as putative tumor-founder cells, these cells remain poorly characterized, in part because methods for their isolation, purification, and expansion are presently not well developed. There is a clear need for such methods, which if successful could provide enriched populations of cancer stem cells that could facilitate better classification and characterization of human brain disorders that involve abnormal behavior of stem cells, and ultimately lead to enhanced diagnosis and treatment options for these aggressive and devastating diseases.

### SUMMARY OF THE INVENTION

The invention addresses some of the deficiencies in the art by generally providing a novel culture paradigm that enables the isolation, expansion, and banking of populations of cancer-derived stem cells. The methods of the invention are exemplified using tissues from brain tumors and other neurological disorders under defined conditions but are equally applicable to isolating and expanding tumorigenic and non-tumorigenic stem cells from other types of tumors. In one embodiment, the methods of the invention apply to solid tumors.

Prior to the invention, stem cell isolation protocols have relied either on the expression of particular cell surface markers or on derivation from previously isolated cells. The invention provides unique methods for separating stem cell populations within tumors, based upon the migratory competence of these cells and their preference to attach to particular molecules of culture substrate at the time of tumor tissue expansion on in vitro. Expansion of distinguishable cell lines can be achieved simultaneously in an array of defined culture conditions under particular conditions favorable for proliferation of stem cells in vitro.

Applied to a series of test specimens obtained during surgery for pediatric brain tumors, the cultures systems and methods of the invention have proven useful for identifying tumors that originate from founder cells with the characteristics of stem cells. Cell populations derived from these tumors by the inventive methods could be expanded and cryo-preserved indefinitely. When engrafted into the brains of mice, some tumor cell lines were shown to be tumorigenic in vivo and to replicate the features of the original brain tumor.

Based upon these discoveries, the invention provides in one aspect a method of isolating a cell population enriched in tumorigenic stem cells. The method comprises at least one and preferably all of the following steps: mincing a tissue sample of a tumor into tissue explants; plating the tissue explants on a substrate coated with a cell-adhesive layer under conditions that promote attachment of the tissue explants and migration of a subpopulation of cells out of the tissue explants onto the adhesive substrate; separating the tissue explants from the migrated cells and dissociating the tissue explants into a single cell suspension, to provide a dissociated cell population and a migratory cell population; and culturing at least one of said cell populations under conditions that promote proliferation of substantially purified tumorigenic stem cells.

Also provided by the invention are isolated cell populations substantially enriched in tumorigenic stem cells derived from a tumor, and isolated clonal cell populations of tumorigenic stem cells derived from these tumors. The disclosed methods of stem cell isolation and culture and the resultant populations of purified cancer stem cells present a wide variety of uses, as further described below.

In certain embodiments, the invention also provides isolated cell populations substantially enriched in tumorigenic stem cells derived from a central nervous system (CNS) tumor. The invention also provides isolated cell populations substantially enriched in non-tumorigenic stem cells derived from a central nervous system (CNS) tumor. In another embodiment, the invention also provides isolated cell populations substantially enriched in tumorigenic cells that do not possess stem cell characteristics derived from a central nervous system (CNS) tumor. The invention also provides isolated clonal cell populations of tumorigenic stem cells derived from a central nervous system (CNS) tumor. Additionally, isolated clonal cell populations of non-tumorigenic stem cells derived from a central nervous system (CNS) tumor. In another embodiment, the invention also provides isolated clonal cell populations of tumorigenic cells that do not possess stem cell characteristics derived from a central nervous system (CNS) tumor.

Another aspect of the invention is a personalized method of treatment of a subject with a tumor, which is made possible by the ready availability and manipulability of cancer stem cells derived from the subject's own tumor using methods in accordance with the invention.

Other aspects and advantages of the invention are discussed below.

### BRIEF DESCRIPTION OF THE DRAWINGS

FIG. 1A is a photograph of a tissue specimen from a human pediatric brain tumor used as the source of tumor cell lines in accordance with invention.

FIG. 1B is a schematic diagram illustrating steps in a standard isolation protocol for generation of cultures containing stem cells from neurospheres.

FIG. 1C is a schematic diagram illustrating steps in a novel adhesive isolation protocol for generation of cancer cell lines from human brain tumors, in accordance with an embodiment of the invention.

FIG. 2A is a series of photographs of neurospheres (NS) at the primary, tertiary and quintary NS stages of cultures derived from tissue samples from three human brain tumors (designated 018-T, 019-T, 020-T).

FIG. 2B is three fluorescence micrographs showing immunostaining of brain tumor cultures shown in FIG. 2A with antibodies against βIII tubulin and GFAP.

FIG. 2C is a graph showing growth characteristics of cultures of brain tumor cell lines 018, 019 and 020 under clonal conditions from 0-360 days in vitro.

FIG. 2D is a graph showing the fraction of sphere-forming cells among the plated cells during culture, from the primary to duodenary NS stages of culture.

FIG. 2E is a graph showing the average number of cells/neurospheres at the indicated stages of NS culture.

FIG. 3 is a graph showing growth characteristics of cultures derived from human brain tumors 001-020, expressed as number of NS (as a percentage number of 1° NS), at various stages of culture from 1° NS to 6° NS. Arrows indicate several cultures that contain self-renewing SFC at the 5° NS and 6° NS stages.

FIG. 4A is three photographs showing histological appearance and immunostaining with GFAP and Ki67 antibodies of original anaplastic ependymoma tumor specimen 018.

FIG. 4B is three photographs showing histological appearance and immunostaining with GFAP and Ki67 antibodies of original glioblastoma multiforme tumor specimen 019.

FIG. 4C is six photographs illustrating appearance in culture at passages 0, 5, and 10 and total cell numbers from 0-70 days in culture for cell lines expanded in defined adhesive conditions from the original tumor specimens 018 and 019 shown in FIGS. 4A and B.

FIG. 4D is two graphs illustrating growth characteristics of adhesive cell populations derived from migrating cells (Mig) and dissociated cells (Diss) grown under several conditions including coating of the growth substrate with laminin/poly-L-omithine (LPO), fibronectin (FN), gelatin (GL), and growth on uncoated plastic (PL).

FIG. 4E is a graph and four photographs illustrating growth characteristics and appearance of cell populations derived from human brain tumor 019.

FIG. 4F depicts the proliferation of migratory cells of FIG. 1c expanded stably for 35 population doublings under adhesive mono-layer conditions.

FIGS. 5A and 5B are five photographs showing histological evidence of tumor formation in a NOD-SCID mouse brain 23 days following engraftment into the brain of cells from 10^{th} passage human cancer cell line 019LPOmig.

FIG. 5C is a still photograph from a movie showing ataxia, freezing and paralysis exhibited by a mouse 39 days after engraftment of human cancer cell line 019LPOmig.

FIG. 6A is eight photographs showing T2-weighted coronal MRI images of the brain of a mouse 44 days after engraftment of human cancer cell line 019LPOmig. The figures show the spread of the tumor across the midline of the consecutive mass effects (stars).

FIG. 6B shows the engraftment of cells from the anaplastic ependymoma case 018LPOmig (cultured under identical conditions as cells in 6A and show to contain stem cells according to the standard neurosphere assay in FIGS. 2 and 3) were unable to reproduce patient-specific tumors even 80 days after engraftment (the arrow demarcates the site of transplantation).

FIGS. 7A-C depict the results of the analysis of mRNA expression profiles of glioblastoma and anaplastic ependymona derived cells. FIG. 7A depicts the lineage of exemplary cell lines. FIG. 7B depicts the results of a tumor formation experiment in an animal model. FIG. 7C depicts the results of a quantatative analysis of the expression of listed mRNA molecules in the identified cell lines.

### DETAILED DESCRIPTION OF THE INVENTION

### Definitions

It is to be understood that the terminology used herein is for the purpose of describing particular embodiments and is not intended to limit the scope of the present invention, which will be limited only by the appended claims. As used herein, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, all terms have the meanings ascribed to them, unless specified otherwise.

### Cancer Stem Cells

A current view in cancer biology is that stem-like cells are present in some human tumors and, although representing a small minority of the total cellular mass of the tumor, are the subpopulation of tumor cells responsible for growth of the tumor. This theory is consistent with clinical observation that certain tumors, for example malignant gliomas, are capable of recurring and/or progressing following conventional surgical and radiation therapy.

The term “stem cell” is known in the art to mean a cell (1) that is a cell capable of generating one or more kinds of progeny with reduced proliferative or developmental potential; (2) that the cell has extensive proliferative capacity; and (3) that the cell is capable of self-renewal or self-maintenance (see, e.g., Potten et al., Development 110: 1001 (1990); U.S. Pat. Nos. 5,750,376, 5,851,832, 5,753,506, 5,589,376, 5,824,489, 5,654,183, 5,693,482, 5,672,499, and 5,849,553, all incorporated by reference). In adult animals, some cells (including cells of the blood, gut, breast ductal system, skin, and neurogenic portions of the CNS referred to as “brain marrow”) are constantly replenished from a small population of stem cells in each tissue. A well-known example of adult cell renewal by the differentiation of stem cells is the hematopoietic system (see, e.g., U.S. Pat. Nos. 5,061,620, 5,087,570, 5,643,741, 5,821,108, 5,914,108, each incorporated by reference). Multipotent stem cells can be isolated from the adult brain and propagated in vitro as described in U.S. Pat. No. 6,638,763, incorporated by reference. Stem cells are also found in other tissues, including epithelial tissues (see, Slack, Science 287: 1431 (2000)) and mesenchymal tissues (see, e.g., U.S. Pat. No. 5,942,225; incorporated by reference). The maintenance of tissues, whether during normal life or in response to injury and disease, depends upon the replenishing of the tissues from stem cells.

In contrast to these normal situations, “tumor stem cells” or “cancer stem cells” are defined as cells that can undergo self-renewal as well as abnormal proliferation and differentiation to form a tumor. Functional features of tumor stem cells are that they are tumorigenic; they can give rise to additional tumorigenic cells by self-renewal; and they can give rise to non-tumorigenic tumor cells. As used herein, particularly in reference to an isolated cell or isolated cell population, the term “tumorigenic” refers to a cell derived from a tumor that is capable of forming a tumor, when dissociated and transplanted into a suitable animal model such as an immunocompromised mouse. A “non-tumorigenic” cell refers to a cell derived from a tumor other tissue that when dissociated, transplanted and tested under identical conditions does not form a tumor in an animal model. Demonstration of the tumorigenicity in vivo of a population of cells derived from a single cloned tumor cell (i.e., a clonal cell line established in vitro from a tumor cell), or apopulation substantially enriched in tumor stem cells provides proof of the concept that a “cancer stem cell” that can give rise to a tumor.

The developmental origin of tumor stem cells can vary among different types of cancers. It is believed that tumor stem cells may arise either as a result of genetic damage that deregulates normal mechanisms of proliferation and differentiation of stem cells (Lapidot et al., Nature 367(6464): 645-8 (1994)), or by the dysregulated proliferation of populations of cells that acquire stem-like properties.

In the stem cell model of tumorigenesis, tumors contain a distinct subset of cells that share the properties of normal stem cells, in that they proliferate extensively or indefinitely and that they efficiently give rise to additional solid tumor stem cells. Within an established tumor, most cells may have lost the ability to proliferate extensively and form new tumors, but tumor stem cells proliferate extensively and give rise to additional tumor stem cells as well as to other tumor cells that lack tumorigenic potential. An additional trait of tumor stem cells is their resistance to therapeutics, such as chemotherapy. It is the small fraction of tumor stem cells and their immediate daughter cell population that proliferates and ultimately proves fatal. In the reality of present medical practice, however, tumors are visualized and initially identified according to their locations, and cytological criteria, not by their developmental origin or by the detection of cells with the attributes of cancer stem cells.

### Isolation, Expansion and Cloning of Cancer Stem Cells

The invention is based on studies of human primary cancers that were isolated from patients, plated as tumor explants, and grown under novel culture conditions that promote natural, cell-initiated separation of cell types within the primary tumor explant into subpopulations, starting from the time of culture initiation. An important aspect of the invention is the discovery that cancer stem cells can be separated from the primary tumor mass by virtue of the propensity of some of these cells to migrate from a tumor explant onto a substrate coated with certain adhesive molecules, allowing for their selective enrichment and propagation. The use of the novel culture techniques and methods of separating subpopulations of tumor cells (starting with tumor tissue explants) distinguishes the invention from previous methods aimed at isolating cancer stem cells, and has provided the means to generate large populations of tumor stem cell-enriched cultures, which by means of subcloning can be substantially freed of non-tumorigenic cells.

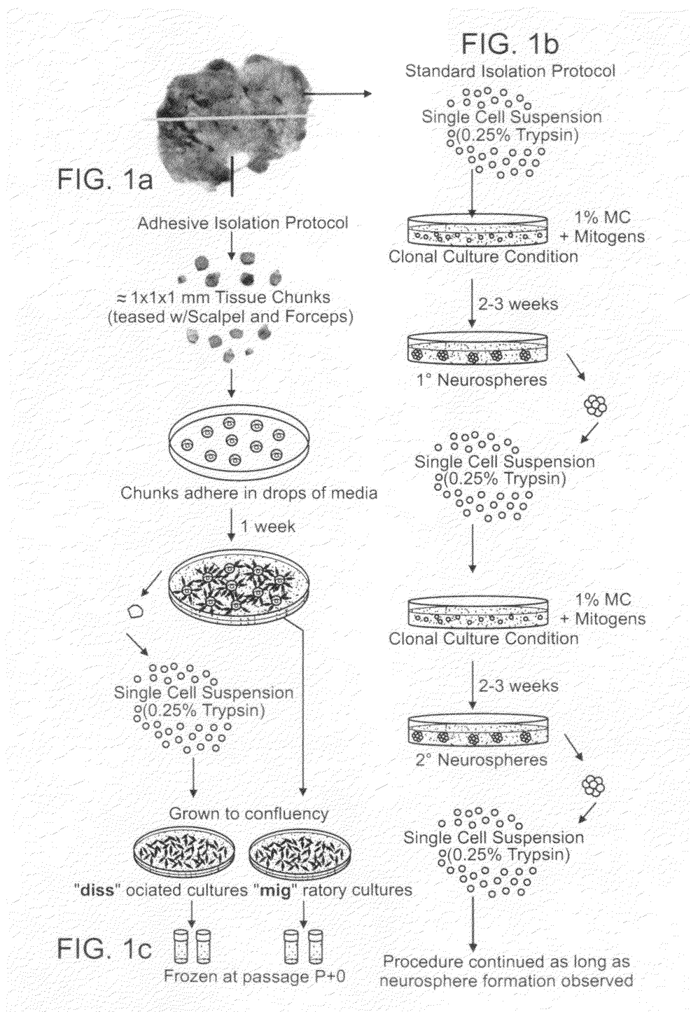

A method of isolating a cell population substantially enriched in tumorigenic stem cells in accordance with the invention is illustrated in FIG. 1C, and comprises at least one or more of the following steps:

(a) mincing a tissue sample from a tumor into tissue explants;

(b) plating the tissue explants on a substrate coated with a cell-adhesive layer under conditions that promote attachment of the tissue explants and migration of a subpopulation of cells out of the tissue explants onto the substrate;

(c) separating the tissue explants from the migrated cells and dissociating the tissue explants into a single cell suspension, to provide a dissociated cell population and a migratory cell population; and

(d) culturing at least one of said cell populations under conditions that promote proliferation of substantially purified tumorigenic stem cells.

Examples of tumors from which tissue samples containing tumor stem cells can be isolated or enriched for according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, mesothelioma, Ewing's tumor, lymphangioendotheliosarcoma, synovioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, astrocytic tumors (e.g., diffuse, infiltrating gliomas, anaplastic astrocytoma, glioblastoma, gliosarcoma, pilocytic astrocytoma, pleomorphic xanthoastrocytoma), oligodendroglial tumors and mixed gliomas (e.g., oligodendroglioma, anaplastic oligodendroglioma, oligoastrocytoma, anaplastic oligoastrocytoma), ependymal tumors (e.g., ependymoma, anaplastic ependymoma, myxopapillary ependymoma, subependymoma), choroid plexus tumors, neuroepithelial tumors of uncertain origin (astroblastoma, chordoid glioma, gliomatosis cerebri), neuronal and mixed-neuronal-glial tumors (e.g., ganglioglioma and gangliocytoma, desmoplastic infantile astrocytoma and ganglioglioma, dysembryoplastic neuroepithelial tumor, central neurocytoma, cerebellar liponeurocytoma, paraganglioglioma), pineal parenchymal tumors, embryonal tumors (medulloepithelioma, ependymoblastoma, medulloblastoma, primitive neuroectodemmal tumor, atypical teratoid/rhabdoid tumor), peripheral neuroblastic tumors, tumors of cranial and peripheral nerves (e.g., schwannoma, neurinofibroma, perineurioma, malignant peripheral nerve sheath tumor), meningeal tumors (e.g., meningeomas, mesenchymal, non-meningothelial tumors, haemangiopericytomas, melanocytic lesions), germ cell tumors, tumors of the sellar region (e.g., craniopharyngioma, granular cell tumor of the neurohypophysis), hemangioblastoma, melanoma, and retinoblastoma. Additionally, the stem cell isolation methods of the invention are applicable to isolating stem cells from tissues other than characterized tumors (e.g., from tissues of diseases such as the so called “stem cell pathologies”).

The term “explant,” as used herein, refers to an isolated portion of a tumor in which the normal relationship of the tissues, cells, and extra cellular matrices within the tumor is left substantially intact and undisturbed to the greatest extent possible following excision of the tumor specimen from the subject. The tumor explants are prepared by gently teasing or cutting the tumor tissue into pieces of suitable size for attachment to tissue culture dishes. For example, tissue pieces measuring about 1 mm³ are suitable for explants of certain brain tumors such as glioblastoma and anaplastic ependymoma.

Successful separation and enrichment of subpopulations of cells from the explant is achieved by plating the tissue explants on a substrate coated with a cell-adhesive layer under conditions that promote attachment of the tissue explants, and in particular, the migration of a subpopulation of cells out of the tissue explants onto the substrate, allowing for their physical separation. The choice of cell-adhesive layer will vary depending upon the type of tumor and the particular characteristics of the migratory cell population, but is selected to promote the natural ability of some cells within the tumor explant to migrate away from the tumor mass. As shown in Examples below, populations of migratory cells isolated in this manner, e.g., from aggressive brain tumors, are highly enriched in neurogenic cancer stem cells. As used herein, the term “neurogenic” refers to a cell having the capacity or propensity to differentiate into one or more cell types of the nervous system or a nervous tissue, including both neuronal cell types and glial cell types. A “neurogenic cancer stem cell” is a stem cell that can undergo self-renewal as well as abnormal proliferation and differentiation to cells expressing markers of neuronal and/or glial cells, and can form a tumor of the CNS.

To prepare a cell-adhesive layer for a culture substrate, the substrate (such as a plastic tissue culture plate or a glass coverslip) is coated with a solution containing laminin and poly-L-omithine (LPO), fibronectin, vitronectin, gelatin, or other suitable mixture. Particular formulations for cell-adhesive layers are described, for example, in Goetz et al. Proc. Natl. Acad. Sci. USA 103(29):11063-11068, 2006 (incorporated by reference), and are provided in Examples below.

Following a suitable time in culture (e.g., about 7-10 days), the tumor explants are removed from the culture dishes, leaving behind the separated migratory cell population attached to the cell-adhesive substrate. Two separate cell populations are prepared at this stage—a “dissociated cell” population, and a “migratory” cell population. The dissociated cell population is prepared by trypinizing the explant to a single cell suspension using standard methods known in the art. Dissociated cell populations can be prepared either from single or combined dissociated explants. Both populations of cells (dissociated and migratory) are then cultured under suitable conditions (migratory cells are plated on the appropriate cell-adhesive coating) until the cultures reach confluency, at which time they may be passaged, and portions of the cells may be cryopreserved for subsequent use.

The tumor-derived stem cells of the invention can be propagated, expanded and passaged extensively in vitro (at least, for example, 15, 20, 25, 30, 35, 40 or more passages) using standard culture conditions. As defined herein, “standard culture conditions” refer to culture conditions suitable for the maintenance and propagation of stem cells without components added to stimulate these cells to differentiate along a particular lineage, for example the neural lineage. Standard culture conditions for cultivating stem cells, including methods for generating clonal cultures have been developed. Preferred methods for isolating and culturing specific embodiments of the cancer stem cells of the invention, including clonal cell lines, are described in detail in the Examples, infra.

One suitable media formulation for derivation of cancer stem cells from human brain tumor explants is termed proliferative media (“P media”), which comprises: DMEM/F12 supplemented with 5% FCS; 100 μg/ml human apo-transferrin (Intergen); 5 μg/ml human insulin (Intergen); 6.29 μg/ml progesterone, 5 ng/ml sodium selenite; 16.1 μg/ml putrescine; 1.1×B27 supplement; 35 μg/ml bovine pituitary extract; 1× antibiotic-antimycotic solution (abx, Invitrogen); and 1,000 units/ml human LIF. EGF and bFGF (each 40 ng/ml) are added the first day of culture, and 20 ng/ml of each are added every other day thereafter.

For in vitro analysis of the multipotency of a suspected cancer stem cell population (multipotency being a hallmark of stem cells), an assay of multipotency is used. For example, for CNS cancer stem cells derived, e.g., from brain tumors, a suitable assay of multipotency is a “standard NS assay,” in which dissociated cells of interest (about 100,000 cells/ml) are distributed in non-adhesive culture dishes in a media formulation (termed “NS media”) comprising: 1% methylcellulose (MC) in DMEM/F12; 5% FCS; modified N2 components (100 μg/ml human apo-transferrin; 5 μg/ml human insulin; 6.29 ng/ml progesterone, 5 ng/ml sodium selenite; 16.1 μg/ml putrescine); 35 μg/ml bovine pituitary extract; 1× antibiotic-antimycotic solution; and 1,000 units/ml human LIF. Epidermal growth factor (EGF) and basic fibroblast growth factor (bFGF), each at concentration of 40 ng/ml, are added the first day, and at 20 ng/ml every other day thereafter. After 2-3 weeks under these conditions, if the cultures contain neurogenic, stem cells, primary neurospheres (NS) will appear and may be counted (e.g., in six wells for each test culture). The primary NS may be collected in plastic tubes, centrifuged at 330 g, and trypsinized for 15 min. After addition of 5% FCS followed by manual dissociation, the cell solution is filtered using nylon mesh to ensure a single cell suspension. The cells are counted, and then distributed at a density of about 50,000 cells/ml in NS media as described above, for derivation of clonal secondary NS. When vital secondary NS arise, higher-degree (tertiary, quaternary, etc.) NS may be prepared at intervals of 2-3 weeks, following the passaging protocol described for primary NS. The steps in the performance of a multipotency assay of cancer stem cells using the standard NS assay are shown diagrammatically in FIG. 1B.

The methods of the invention may further comprise analyzing the cellular markers of the isolated stem cells. For example, for analysis of cellular markers of multipotency, cancer stem cell-derived NS may be attached to LPO-coated glass coverslips and maintained without growth factors for 14-35 days in Neurobasal™ medium (Invitrogen, Carlsbad, Calif.) containing 1×B27 supplement, 2 mM L-glutamine and antibiotics as described. Cells are then fixed, e.g., in ice-cold 4% paraformaldehyde for 20 minutes in preparation for immunohistochemical detection of markers of neuronal and glial lineage. Suitable lineage markers and techniques for their detection are known in the art and are described in detail, for example, in Scheffler et al., Proc. Natl. Acad. Sci. 102(26):9353-9358, 2005 and in Examples, infra. Alternatively or additionally, lineage markers specific for any tissue of origin of a tumor, and other tumor markers may be detected by immunocytochemistry or, e.g., by flow cytometry using suitable antibodies using techniques well known in the art.

The tumorigenicity of a cancer stem cell isolated by the methods of the invention can be confirmed by demonstration of tumor growth in a suitable host animal. The host animal can be a model organism such as nematode, fruit fly, zebrafish; preferably a laboratory mammal such as a mouse (nude mouse, SCID mouse, NOD/SCID mouse, Beige/SCID mouse, FOX/SCID mouse), rat, rabbit, or primate. Severely immunodeficient NOD-SCID mice are particularly suitable animal recipients of transplanted human cancer stem cells. Immunodeficient mice do not reject human tissues, and SCID and NOD-SCID mice have been characterized as hosts for in vivo studies of human hematopoiesis and tissue engraftment. McCune et al., Science 241: 1632-9 (1988); Kamel-Reid & Dick, Science 242: 1706-9 (1988); Larochelle et al., Nat. Med. 2: 1329-37 (1996). In addition, Beige/SCID mice can be used. The NOD/SCID or Beige/SCID mice can be further immunosuppressed, using VP-16, radiation therapy, chemotherapy, or other immunosuppressive biological agents.

Typically, single-cell suspensions (or suspensions with a few aggregates of cells, such as 20,000 cells; ideally less than 100; preferably less than 10 cells) are prepared from the isolated cancer stem cells and transplanted into appropriate anatomical sites in the mice. General techniques for formulation and injection of cells may be found in Remington's Pharmaceutical Sciences, 20th ed. (Mack Publishing Co., Easton, Pa.). Suitable routes may include parenteral delivery, including intramuscular, subcutaneous, intramedullary injections, as well as intrathecal, direct intraventricular, intravenous, intraperitoneal, intranasal, intracerebral, or intraocular injections, for example. For injection, the cells of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art. A detailed procedure for transplantation into the brain and analysis of the tumorigenicity of cancer stem cell lines derived from brain tumors is described in detail in the Examples, infra. The in vivo assay is useful for initial verification of the tumorigenicity of a tumor-derived stem cell line. Once tumorigenicity is established, the animal model can be used for a wide array of biological and molecular assays to characterize the tumorigenic stem cells and the tumors that arise therefrom.

### Methods of Use of Isolated Cancer Stem Cells

The disclosure herein provides documented evidence in support of proposed models of tumorigenesis by cancer stem cells. Importantly, the invention provides methods for rapid isolation and propagation of cancer stem cells, and isolated populations of cancer stem cells of immediate practical use in designing and testing targeted therapeutic strategies aimed at killing or slowing the proliferation of cancer stem cells, which are at the heart of the malignant disease mechanism.

By this invention, it is contemplated that discovery of effective methods of treatment for these disorders will be rapidly advanced, by virtue of the availability of nearly unlimited numbers of cell populations substantially enriched in tumorigenic stem cells, which are relatively rare in tumor tissue as a whole. One major advantage of the disclosed methods of isolating cancer stem cells from tumors is that it is now possible to isolate, expand, cryo-preserve and bank distinct tumor cell populations substantially enriched in tumorigenic stem cells derived from tumors of individual patients. “Enriched,” as in an enriched population of cells, can be defined based upon a functional characteristic such as tumorigenic activity, e.g., the minimum number of cells that form tumors at limiting dilution in test mice. Thus, e.g., if 500 tumor stem cells form tumors in 80% of test animals, but 5000 dissociated cells from a tumor are required to form tumors in 80% of test animals, then the tumor stem cell population is 10-fold enriched for tumorigenic activity.

As discussed, and shown in Examples below, it is also possible to create clonal cell lines derived from tumors using the tumor stem cell isolation method described herein. Cell lines of the invention are capable of reproducing the tumor of origin in an animal model.

In one embodiment, the efficiency of the tumor stem cell isolation methods on adhesive substrates as described herein represents an increase of 130-230% as compared to a standard neurosphere assay for isolation of neurogenic stem cells. Remarkably, clonal cell lines established from such adhesive cultures represent a substantial (e.g., about 55-fold) enrichment over cell populations made using the neurosphere assay.

As will be apparent to those of skill in the art, the methods and compositions of the invention provide greatly enhanced opportunities for diagnosis of tumors from patients. The database of diagnostic information will continue to expand as more and more markers are discovered through banking of cryo-preserved lines of tumor stem cells made possible by the invention, and dissemination of the cells to research and medical facilities around the world. The additional knowledge gained from comparisons of tumorigenic stem cell lines derived from multiple patients should be a substantial addition to the criteria currently used, for example to diagnose brain tumors, under the World Health Organization grading scale. Information gained from analysis of unique cancer markers in the isolated and characterized cells is expected to provide the basis for more precise characterization, and even reclassification of certain tumors.

Accordingly, another aspect of the invention is a method of classifying a tumor comprising cancer stem cells. The method includes one or more of the following steps:

(a) obtaining a tissue sample of a tumor from a subject;

(b) culturing at least one cell population substantially enriched in cancer stem cells derived from the subject's tumor;

(c) identifying one or more biological markers in the cancer stem cells that are expressed at different levels in the stem cells as compared to non-tumorigenic cells of the tumor; and

(d) classifying the stem cell or tumor on the basis of the presence, or relative proportion, of the biological markers of stem cells, as compared with the presence or proportion of said biological markers in other tumors, and in normal control tissues.

In exemplary embodiments, in their undifferentiated state, isolated cells of the tumor stem cell lines are multipotent stem-like cells that can, upon stimulation (withdrawal of mitogens), differentiate into GFAP+(a marker of the glial cell lineage) and βIII tubulin+(a marker of the neuronal cell lineage) cells. A hierarchical progression of the tumor stem cells from immature to more differentiated phenotypes is predicted, and can be analyzed by evaluating expression of a battery of markers by methods known in the art, for example by evaluating marker expression at various stages of differentiation in vitro under defined conditions, or in tumors formed by these cells at various intervals after administration in vivo.

Using a defined culture system in accordance with the invention, long-term self-renewing stem cells can be reliably isolated from brain tumors, expanded in vitro, and banked for future analysis. In fact, one brain tumor may contain several biologically distinct stem cell populations with tumorogenic potential, as shown in a case of glioblastoma multiforme. Of note, in some cases non-tumorigenic stem cells can also be isolated from brain tumor tissue and propagated, as exemplified by a cell line derived from a case with anaplastic ependymoma, described infra.

As discussed, characteristic antigenic profiles of biologically distinct cell populations among different brain cancer cell lines, or among different clones within individual tumor cell lines can be determined, e.g., using antigenic markers well known to those of skill in the art. The availability of tumorigenic cell lines for such characterization may enable detection of cells expressing particular markers in vivo, allowing for their recognition and potentially direct extraction from tumor tissue from patients in need thereof.

To aid in the discovery of effective new therapeutic agents against brain tumors, the tumor cell lines of the invention can be used in molecular profiling studies using comparative cancer microarrays (described, e.g., in Segal et al., 2005) or comparative microRNA analysis (see, e.g., Hammond, 2006; Cheng et al., 2006). Such approaches can be used to uncover common genetic traits and intracellular pathways that could be targeted to circumvent the resistance to currently available therapeutics of pathologies derived from malignant stem cells.

The ability to isolate and rapidly expand a population of cancer stem cells from a patient's tumor using the methods of the invention further offers the exciting possibility for personalized medicine for patients suffering from tumors, for example involving testing candidate therapeutic compounds on the patient's own malignant tumor cells, following surgical removal of the tumor and during the subsequent course of treatment. Thus, another aspect of the invention is a method of treatment of a subject with a tumor comprising:

(a) obtaining a tissue sample of the tumor from the subject;

(b) culturing at least one cell population substantially enriched in tumorigenic stem cells derived from the subject's tumor according to the methods described herein;

(c) identifying an effective therapeutic agent or method to kill or delay the growth of the subject's tumorigenic stem cells; and

(d) administering the effective therapeutic method or agent to the subject to prevent or delay the growth of the tumor. It is expected that the cells of the invention will be invaluable tools for screening assays such as high throughput assays of potentially effective cytotoxic agents, for example.

The invention is further illustrated by reference to the following non-limiting examples.

### EXAMPLES

### Example 1

### Materials and Methods

The following materials and methods are generally useful to carry out the invention, and were used as needed to conduct studies outlined in the Examples.

Derivation of Cells and Methods of Culturing.

Human tissue derived from brain surgery was transferred to the laboratory following institutional approved protocols. Brain tissue was minced under sterile conditions into chunks of about 5 mm³ (illustrated in FIG. 1A), and randomly divided into two equal parts. One part was fixed in 4% paraformaldehyde and stored until further use for histological analysis; the other part was further minced to 1 mm³-sized chunks of vital tissue, which was used for in vitro preparations.

The steps in preparation of standard neurosphere (NS) cultures (assays) is shown schematically in FIG. 1B. Vital brain tissue as described above was placed in a 0.25% trypsin solution on a shaker overnight at 4° C. Five percent fetal calf serum (FCS, HyClone) was added, and the next day the tissue chunks were gently dissociated manually into a single cell suspension using graded fire-polished glass pipettes. Trypan blue exclusion was used to confirm viability of the cells. Dissociated cells (100,000 cells/ml) were distributed in non-adhesive culture dishes (Corning) in a media mixture (termed “NS media”) comprising: 1% methylcellulose (MC) in DMEM/F12; 5% FCS; N2 components; 35 μg/ml bovine pituitary extract; 1× antibiotic-antimycotic solution (abx, Invitrogen); and 1,000 units/ml human LIF (Chemicon). Epidermal growth factor (EGF) and basic fibroblast growth factor (bFGF), each at concentration of 40 ng/ml, were added the first day, and at 20 ng/ml every other day thereafter. Unless otherwise specified, media and growth factors were purchased from Sigma, Invitrogen, and R&D systems.

After 2-3 weeks, primary neurospheres (NS) were counted (6 wells for each experiment), collected in 15 ml Falcon tubes, centrifuged at 330 g, and trypsinized for 15 min. Five percent FCS was added, followed by manual dissociation as described above. The cell solution was filtered using 70 μm nylon meshes (Falcon) to ensure single cell suspension. Cells were counted, and distributed at a density of 50,000 cells/ml in NS media as described above, for derivation of clonal secondary NS. When vital NS arose, higher-degree (tertiary, quaternary, etc.) NS were prepared at intervals of 2-3 weeks by following the passaging protocol described above for primary NS (FIG. 1B).

In various protocols, cells were plated on glass coverslips that had been previously coated using one of the following methods. Laminin/poly-L-ornithine (LPO) coating was performed by incubating coverslips in 140 μl/cm² poly-L-ornithine solution (15 μg/ml; #P-3655, Sigma) at 37° C. for 96 hr followed by washes in Ca⁺⁺/Mg⁺⁺-free PBS and in DMEM/F12 before cells were plated in the presence of 1 μg/ml laminin-1 (#23017-015; Invitrogen). Fibronectin (FN) coating was carried out with 50 μl/cm² fibronectin (50 μg/ml; #33010-018, Invitrogen) at 37 degrees for 1 hr. Gelatin (GL) coating was performed for 30 min at room temperature using 140 μl/cm² gelatin (0.1%; #G-1890, Sigma). Gelatin was removed prior to plating of cells. Dishes were washed three times in DMEM/IF12 before cell seeding.

For analysis of multipotency, NS derived from the ‘standard NS assay’ were attached to Laminin/poly-L-ornithine (LPO)-coated glass coverslips and maintained without growth factors for 14-35 days in Neurobasal™ medium (Invitrogen, Carlsbad, Calif.) containing 1×B27 supplement, 2 mM L-glutamine and abx (Invitrogen). Cells were fixed in ice-cold 4% paraformaldehyde for 20 minutes.

For preparation of “adhesive cultures” from tumors, the remaining half of vital tumor tissue chunks as described above were further teased into the smallest-possible fragments using a scalpel and forceps. Approximately 50 of these micro-fragments were placed into 6 cm culture dishes (Corning) in individual drops (100 μl) of media containing 10% FCS in DMEM/F12+abx overnight. In addition to standard, uncoated plastic dishes (Corning) (“PL”), three defined surface coatings (i.e., “LPO,” “VN,” and “GL”) were used for the simultaneous preparation of adherent cultures at this step. During the course of one week, the volume of media around adherent micro-fragments was gently increased to a total of 4 ml/6 cm dish, by daily additions of proliferative media (“P media”) consisting of: DMEM/F12 supplemented with 5% FCS; 100 μg/ml human apo-transferrin (Intergen); 5 μg/ml human insulin (Intergen); 6.29 ng/ml progesterone, 5 ng/ml sodium selenite; 16.1 μg/ml putrescine; 1.1×B27 supplement; 35 μg/ml bovine pituitary extract; 1×abx; and 1,000 units/ml human LIE EGF and bFGF (each 40 ng/ml) were added the first day, and 20 ng/ml of each were added every other day thereafter.

After 7-10 days, cells were observed to migrate out and proliferate around the vicinity of the adhesive tissue chunks. All adhesive tissue fragments (containing cells that were unable to migrate out onto the respective culture dish surface) were removed at this time, trypsinized, and distributed in P media into one 6 cm dish coated with the same substrate. Thus, for each adhesive condition, two types of culture preparations were made—one containing migratory active cells (“mig”), the other containing dissociated migratory-inactive, resident cells (“diss”) from the same tissue specimen (FIG. 1C). Every other day, 20 ng/ml EGF and bFGF were added; and P media was changed every four days; 1 μg/ml laminin-1 was continuously present in LPO conditions.

For banking of derived cell lines, cells from all conditions were grown to confluency and frozen without further passaging (P+0) in two cryotubes per 6 cm culture dish (i.e., approximately 250,000 cells/tube) (FIG. 1C).

Expansion and Analysis of Adhesive Cell Populations.

For comparative analysis of condition-specific proliferative capacity, cells from one cryotube were plated into one 6 cm dish coated with the respective adhesive substrate, and expanded in P media supplemented with EGF and bFGF as described above. Cells were grown to confluency, trypsinized, counted, and passaged in ratios of 1:2 for up to 20 passages. Numbers of population doublings (PD) were determined using Hayflick's formula (1973): n=3.32(log UCY−log 1)+X, where (n) is the final PD number at end of a given subculture; (UCY) is the cell yield at that point; (I) is the cell number used as inoculum to begin that subculture; and (X) is the doubling level of the inoculum used to initiate the subculture being quantified. The ratio of time spent between passages and PD number was used to estimate cell cycle times for expanding adhesive cell populations. Cells were documented photographically at every passage using a Leica DM IRB microscope and a Leica DFC 300F camera system with included software.

At passages 5, 10, and 20, expanded cell populations were investigated for presence of neurosphere forming cells (NSFC) using the standard NS assay described above. Condition-specific NS were attached to plastic dishes coated with the respective substrate for analysis of multipotency, and otherwise processed as described for the standard NS assay.

Clonal cell lines were derived from selected adhesive cell populations at passage 5 by plating 2-20 cells/cm² in a substrate-coated 10 cm plastic dish (Corning). Colonies could be visually identified at 30-60 days after plating, and were selected and trypsinized using 8 mm cloning rings (Corning). Expansion of clones and analysis in the standard NS assay was performed as described above for substrate-specific adhesive cell populations.

Immunocytochemistry.

The basic immunolabeling buffer contained PBS, 10% FCS, and, for intracellular antigens, additionally 0.1% Triton X-100. After blocking nonspecific antibody activity for 20 min in 5% goat serum, primary antibodies (βIII tubulin, monoclonal mouse, 1:3000, Promega; GFAP, polyclonal rabbit, 1:400, DAKO; CNPase, monoclonal mouse, 1:250, Chemicon) were applied for 4 hours at room temperature. Antigens were visualized using corresponding secondary antibodies (Jackson ImmunoResearch, West Grove, Pa., or Molecular Probes, Eugene, Oreg.). Cell nuclei were labeled for 10 min with 0.8 μg/ml DAPI (Sigma). Fluorescence microscopy was performed on a Leica DMLB upright microscope (Leica, Bannockbum, Ill.) and images were captured with a Spot RT Color CCD camera (Diagnostic Instruments, Sterling Heights, Mich.).

RNA Extraction and RT-PCR Analysis.

Total cellular RNA was isolated from distinct NS and adhesive cell samples using the RNeasy Mini Kit following the manufacturer's recommendations (Qiagen, Valencia, Calif.) and processed for comparative analysis on RNA microarrays and micro RNA gene chips (Illumina).

Transplantation and Analysis of Tumor Formation.

All animal experimentation was conducted according to institutional IACUC guidelines. Cells were trypsinized and concentrated to a density of 20−25×10³/μl DMEM/F12, and one μl of cell suspension was injected into either the lateral ventricle or frontal cortex of adult (>90 days) immuno-compromised NOD-SCID mice (Charles River) (n=8 per experiment). The stereotactic coordinates were: 2.5/−0.5/1.2 and 1.5/2/1.3 (mm depth, A-P, lateral), respectively. Behavioral abnormalities of animals developing tumor-related signs were videotaped.

Animals were sacrificed and perfused transcardially with 4% paraformaldehyde. Brains were removed and stored in 2% paraformaldehyde until further use. MRI data were obtained using an 11-T magnet from fixed brains samples placed in PBS for the imaging session. For histological analysis and standard H&E staining, brains were placed in 2% paraformaldehyde containing 30% sucrose (v/v) overnight, sectioned into 20 μm coronal sections on a freezing microtome, and stored in cryoprotectant.

### Example 2

### Preparation of Cell Lines from Human Pediatric Brain Tumors Using Standard Neurosphere Assay Conditions

In an initial study, tissue samples were randomly collected from pediatric brain tumors over the course of one year. The tumors were located in a broad range of CNS locations, and histopathological diagnosis ranged from WHO scale II (slow growing tumor) through IV (highly malignant, fast growing). Five additional samples that served as a control group for this study were either diagnosed as not of tumor origin, or represented tumors not primarily originating from CNS tissue (Table 1, infra).

TABLE 1
Description of cases and tissue samples used for analysis.

| Case | Age/Sex | Location | Pathologic Diagnosis |
|---|---|---|---|
| 001 | 6/F | 4^{th} ventricle, cerebellar-pontine angle | Ependymoma w/“classic” histology. |
| 002 | 10 mt/F | Suprasellar, w/optic apparatus involved | Pilomyxoid astrocytoma. Uniform, piloid-microcystic. |
| 003 | 3/F | 4th ventricle/posterior fossa | PNET/medulloblastoma w/“classic” histology. |
| 004 | 6/F | Superior vermis/pineal/tectal plate mass | PNET/medulloblastoma w/“classic” histology. |
| 005 | 2/F | (Dys): Frontal insular, dysplastic cortex | Cortical dysplasia. |
| | | (Cx): Lateral, epileptogenic cortex | |
| 006 | 4/F | Large left-temporal tumor | Recurrent GBM w/mostly small cell-, some larger eosinophilic cell-areas. |
| 007 | 6/F | Left frontal lobe, thalamus, basal ganglia | Low-grade glial neoplasm with extensive calcification. |
| 008 | 6/F | (a): L3-S1 metastasis of (b) | Anaplastic ependymoma. |
| | | (b): Recurrent 4^{th} ventricular tumour | |
| 009 | 5/M | Periventricular (EP) temporal lobe, and (HC) | No tumor, no dysplasia. |
| | | hippocampus tissue, hemispherectomy | |
| 010 | 8/F | Right occipital, superficial lesion | Dysembryoplastic neuroepithelial tumor. |
| 011 | 17/M | 4^{th} ventricular mass | PNET/medulloblastoma w/“classic” histology. |
| 012 | 7/M | Right fronto-parietal w/leptomeninx mets. | Anaplastic PXA (third recurrence of original non-anaplastic PXA). |
| 013 | 60/F | Temporal lobe cortex from epilepsy surgery | No significant pathology in cortex and subcortical white matter, no AHS. |
| 014 | 21/F | Left cerebellar, cystic mural tumor nodule | Hemangioblastoma. |
| 015 | 13/F | Intraventricular mass | Pilocytic astrocytoma, rare foci w/nuclear atypia, rare mitoses. |
| 016 | 3 d/F | Surprasellar tumor w/hydrocephalus | Craniopharyngioma. |
| 017 | 11/M | Left medial-inferior frontal lobe mass | Cerebral PNET, densely cellular, mitotically active tumor. |
| 018 | 12/F | Cystic, left occipital lobe mass | Anaplastic ependymoma, clear cell type, high mitotic activity. |
| 019 | 9/M | Hypothalamic, third ventricular neoplasm | GBM (Recurrence, original pilomyxoid histology with high mitotic activity. |
| 020 | 18/M | Midline posterior fossa within 4^{th} ventricle. | PNET/medulloblastoma w/“classic” histology |

Tissue samples from cases 005, 009, 013 (adult epilepsy surgery), 014, and 016 were either not diagnosed as tumors or represent tumors not primarily originating from CNS tissue, thus, these samples served as control for our study. “Classic histology” of medulloblastoma-diagnosis refers to absence of desmoplasia or anaplastic features.
Abbreviations: AHS, Ammon's horn sclerosis; Cx, cortex; d, days; Dys, dysplasia; GBM, glioblastoma multiforme; L1-S3, spinal cord levels; mets, metastases; mt, months; PNET, primitive neuroectodermal tumor; PXA, pleomorphic xanthoastrocytoma

Since the early 1990's, the standard isolation protocol for CNS stem cells has been the neurosphere (NS) assay. The NS assay enables analysis of the key characteristics that define stem cells: i.e., proliferation, self-renewal, and multipotency in appropriate culture conditions (Reynolds and Weiss, 1992; Kukekov et al., 1999; Reynolds and Rietze, 2005). Applied to our group of brain pathology cases, we found only a minority of tissue samples that contained long-term self-renewing stem cell populations.

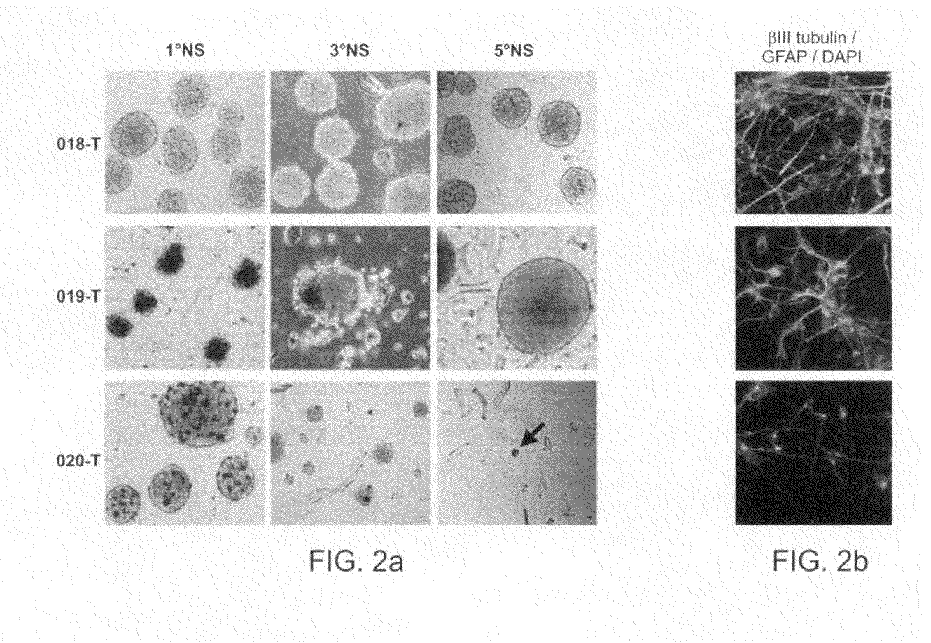

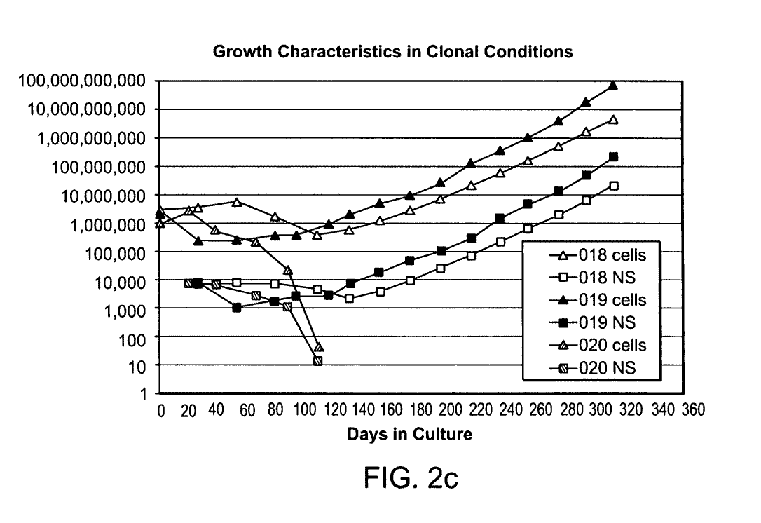

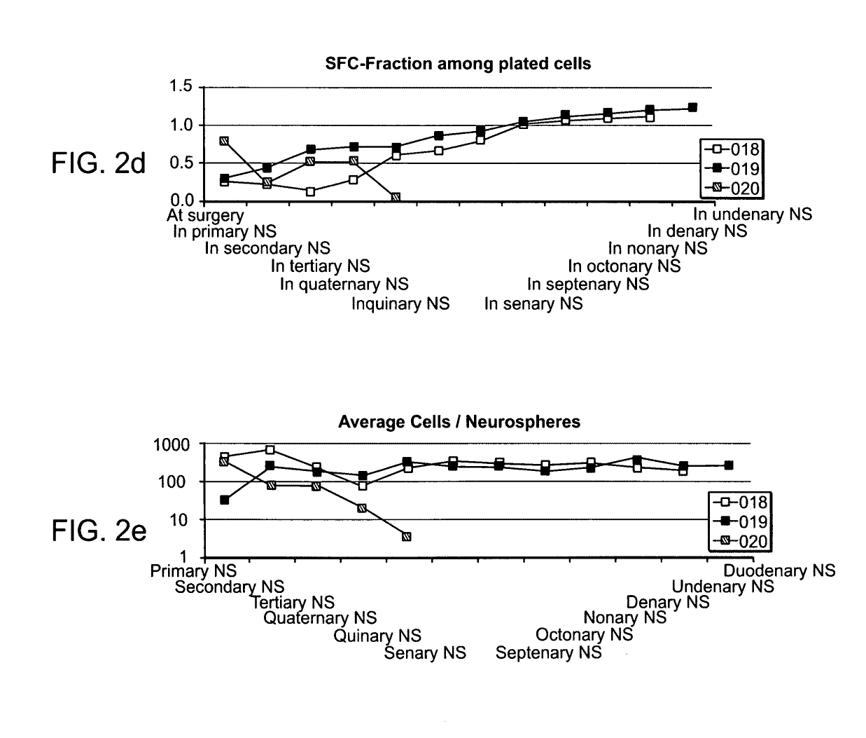

Evaluation for the presence of stem cells using the standard NS assay demonstrated that these cases showed a high incidence of neurosphere (NS)-forming cells (FIG. 2A). NSFC: represent multipotent stem cells that can clonally proliferate (into NS) in non-adhesive conditions, and which, upon plating of NS, can differentiate into neuronal (exemplified by βIII tubulin-expression) and glial (exemplified by GFAP-expression) phenotypes, as shown in FIG. 2B. The cells were capable of self-renewal and clonal expansion for long periods of time, as demonstrated by the fact that upon dissociation of NS to a single cell suspension, increasing numbers of higher-degree NS were formed for more than 16 passages (nearly one year in culture) (FIG. 2C). However, with increasing time in culture, NSFC fractions increased only slowly (FIG. 2D), and the number of cells per NS stabilized (FIG. 2E), indicating an assay-bound equilibrium of NSFC proliferation.

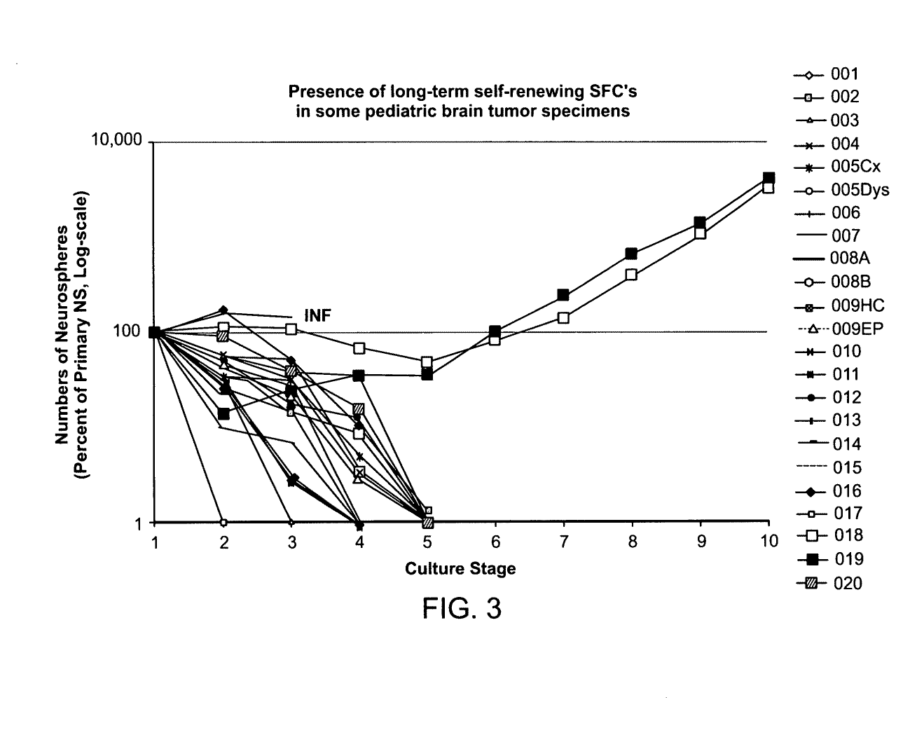

The standard NS assay is well suited to identifying tissue samples containing long-term self-renewing stem cell populations (FIG. 3). More specifically, FIG. 3 summarizes analyses of the numbers of NS formed (as a percentage of primary NS) at various stages of tissue culture (up to 6° NS), in cultures derived from 21 tissue specimens from pediatric brain tumors and evaluated using the standard isolation protocol illustrated in FIG. 1B. In the results shown in FIG. 3, cases indicated as 001 and 004 were not analyzed, and analysis of case 008a was terminated due to contamination at the tertiary NS stage.

In each case, primary neurospheres (1° NS) appeared. However, only a few specimens contained neurosphere-forming cells (NSFC) that continued to produce clonal NS beyond the stage of quaternary NS in vitro (see, e.g., cultures 018 and 019, indicated by arrows, in FIG. 3).

### Example 3

### Clonal Cancer Stem Cell Lines Derived from Human Pediatric Brain Tumors

The standard NS assay, although suitable for identifying tissue samples comprising self-renewing stem cells as shown in the above Example, is limited in its usefulness for purposes of purification, expansion, and detailed study of putative stem cell populations by numerous factors including the slow increase of total cell numbers and low ratios of NSFC to other non-tumorigenic cells; the lengthy passage time (2-3 weeks); and the uncontrollable environment that exists within neurospheres. This study was undertaken to evaluate surface coatings for cell culture dishes that could be useful for isolating, maintaining and expanding human brain tumor-derived cancer stem cells under controlled conditions.

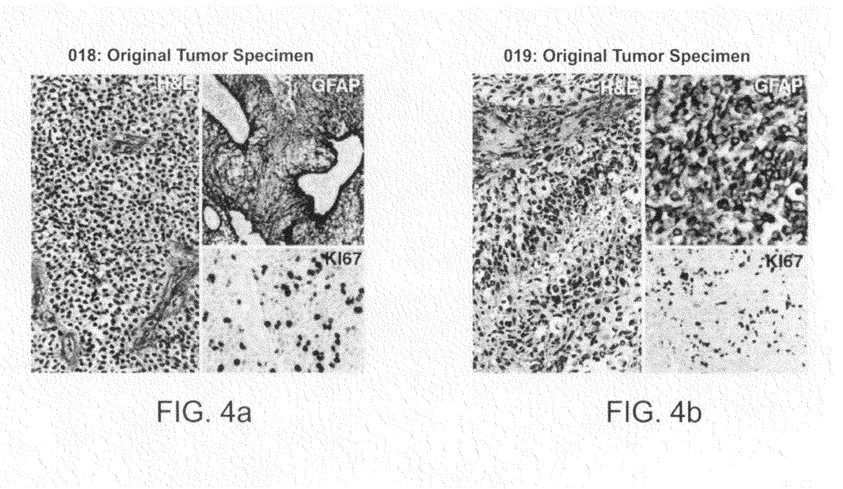

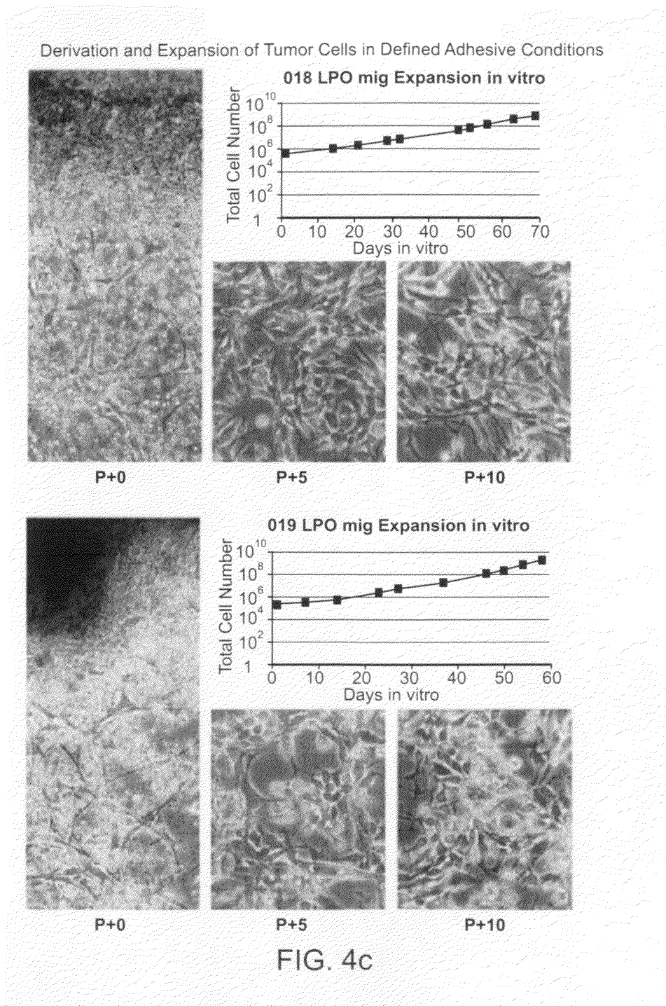

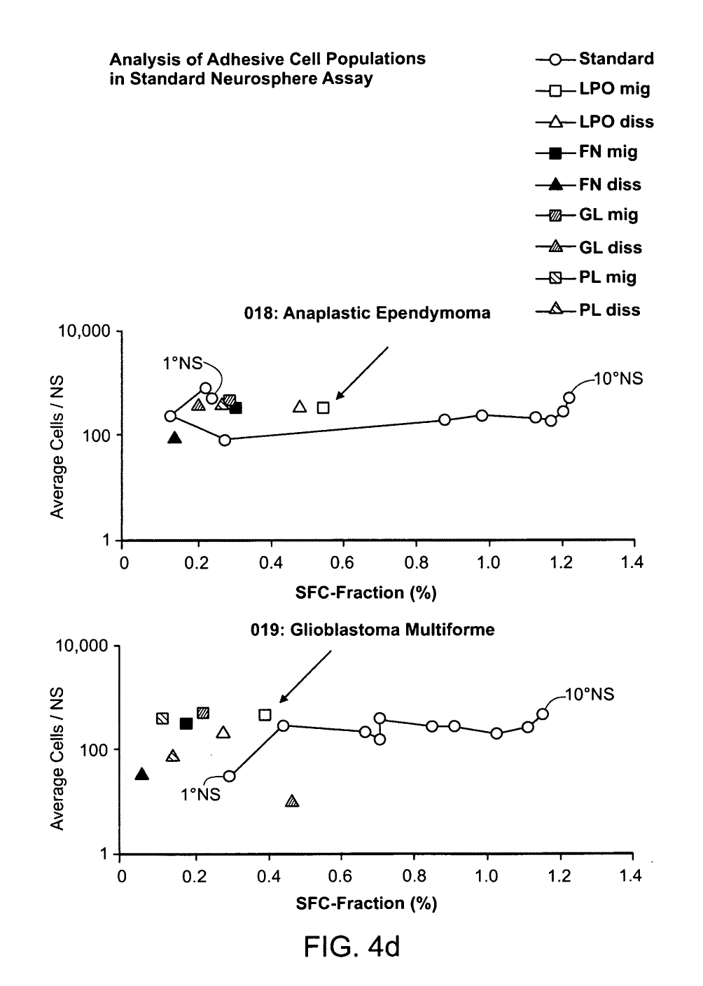

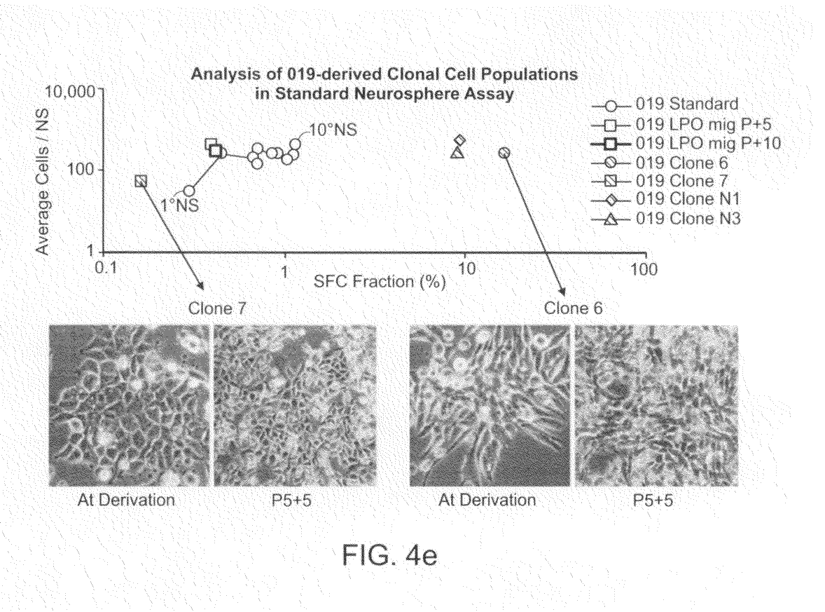

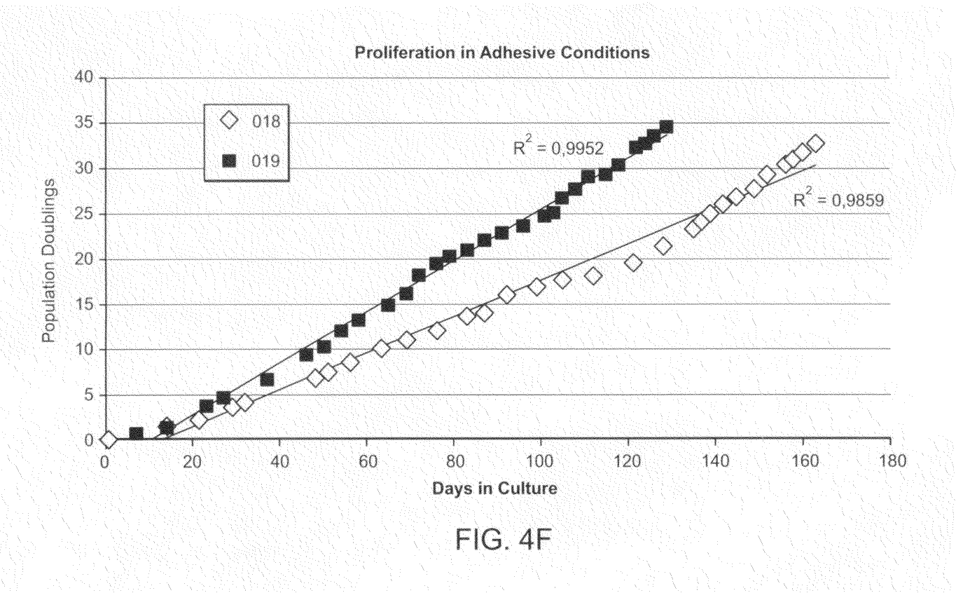

A total of eight different cell populations was separated, based on their migratory competence and preference to attach to distinct substrates at the time of tissue extraction, using procedures as described above in Methods (Example 1). These populations were derived from the tumor specimens 018 and 019, which were respectively diagnosed as anaplastic ependymoma (AEp) and glioblastoma multiforme (GBM) (Table 1). FIG. 4A shows the histological appearance and immunostaining with antibodies against GFAP and Ki67 (the latter being a marker of mitotically active cells) of the original tumor specimen (AEp) of case 018; FIG. 4B shows the corresponding images for the GBM case 019.

In both cases, it was determined that adhesive cell populations could be expanded stably over prolonged periods of time, and could be propagated at least up to 20 passages, corresponding to 25±5 population doublings (PD), without any obvious signs of senescence or change in morphology (FIG. 4C). The calculated cell cycle times varied markedly between individual substrate-specific cell populations (157-322 hours for case 018, and 111-236 hours for case 019, respectively). The following adhesive condition-specific cell cycle times were determined for case 018 (in hours): LPOmig=183; LPOdiss=322; FNmig=157; FNdiss=275; GLmig=179; GLdiss=271; PLmig=n.d. (no initial outgrowth of cells on plastic culture dish surfaces); PLdiss=244. The following condition-specific cell cycle times were determined for case 019 (in hours): LPOmig=117; LPOdiss=158; FNmig=184; FNdiss=111; GLmig=123; GLdiss=123; PLmig=128; PLdiss=236. From these results it is apparent that the cell cycle times for the migratory populations from the 018 case were significantly shorter than those of the dissociated cell populations on each adhesive substrate; however such a consistent pattern was not observed for the cell lines derived from the 019 case, although in both cases migratory cells plated on LPO had significantly shorter cell cycle times than the corresponding dissociated cells from the tumor explants.

At passage 5 (3-7 PD), substrate-specific cell populations were analyzed for NSFC presence and activity (FIG. 4D). Unexpectedly, migratory cells derived on laminin/poly-L-omithine-coated culture dishes (LPOmig populations) contained the highest numbers of NSFC, outperforming the isolation efficacy of the standard NS assay for both brain tumor specimens. This result indicated that the novel adhesive LPOmig conditions are well suited for reliable isolation, rapid expansion (6× faster compared to standard NS assay), and banking of NSFC under defined conditions.

An additional advantage of the inventive method is the feasibility under these conditions to propagate clonal cell-derived cell lines. A cell line derived from the migratory cells from the 018 Epa case grown under adhesive conditions on LPO-coated surfaces was designated 018 LPOmig, and a cell line derived from migratory cells from the 019 GBM case grown under the same conditions was designated 019LPOmig.

Preliminary data from this study additionally indicates the presence of heterogeneous stem cell populations present in GBM tissue (FIG. 4E), providing additional explanation for resistance of these tumors to chemotherapy (Dean et al., 2005).

### Example 4

### Tumorigenicity of Clonal Cancer Stem Cells

It is generally assumed that, under the right conditions, cancer cell lines can retain the properties of the cancers of origin (Masters, 2000; Lee et al., 2006). To determine whether NSFC-rich LPOmig populations as described in the above Example could reproduce the characteristic disease phenotypes of GBM and AEp, cells were engrafted after 10 passages in vitro (corresponding to 12 and 14 PD, respectively) into recipient adult mouse brains. For these experiments, 8 animals were injected with 2−2.5×10³ of 018 LPOmig or 019LPOmig cells into either the lateral ventricle (n=4) or the frontal cortex (n=4).

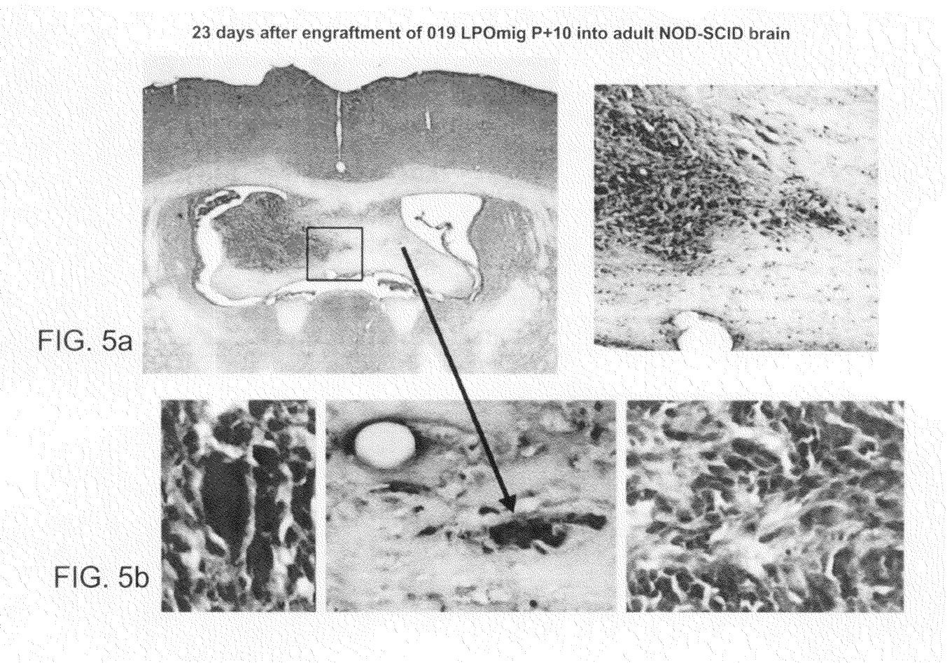

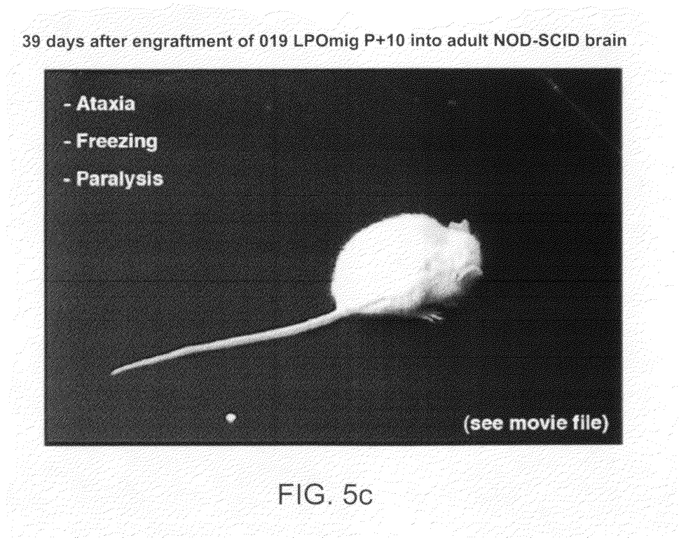

Surprisingly, none of the AEp-derived cell injections yielded the formation of ependymoma-like tissue (evaluated up to 80 days after transplantation), whereas all of the GBM-derived cell grafts formed tumors with histological features closely resembling the original cancer. At three weeks after engraftment, dense clusters of proliferative active donor cells were observed, with individual tumor cells infiltrating the surrounding host tissue, and also traveling considerable distances across the midline (FIG. 5A). Multinucleated tumor giant cells were observed among the donor cell population—a characteristic finding also observed in the original GBM (FIG. 5B). After 5-6 weeks, all animals exhibited similar neuro-behavioral abnormalities, including sudden freezing of motion, strong ataxia, and plegic gait (FIG. 5C), which was apparent in video images. These signs could be directly correlated with a massive donor cell proliferation and spread of tumor cells throughout the recipient brain.

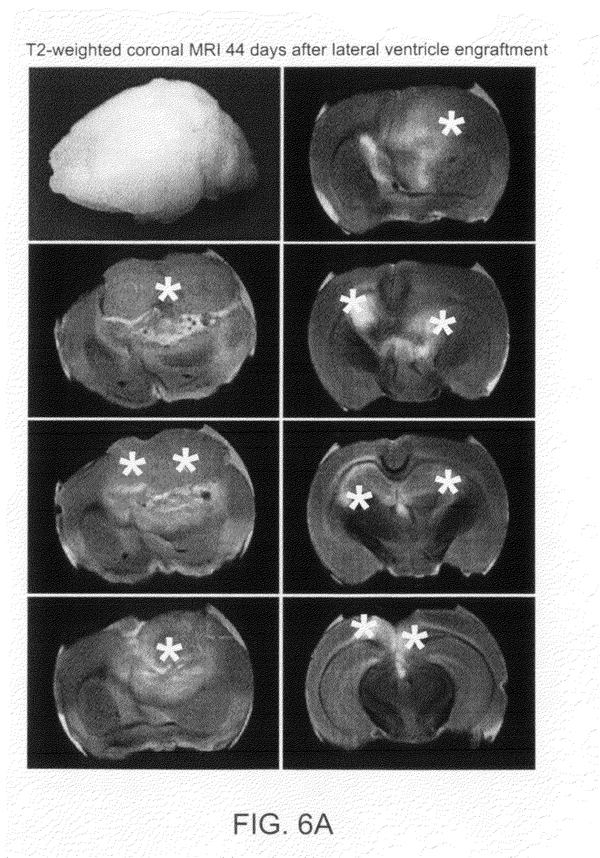

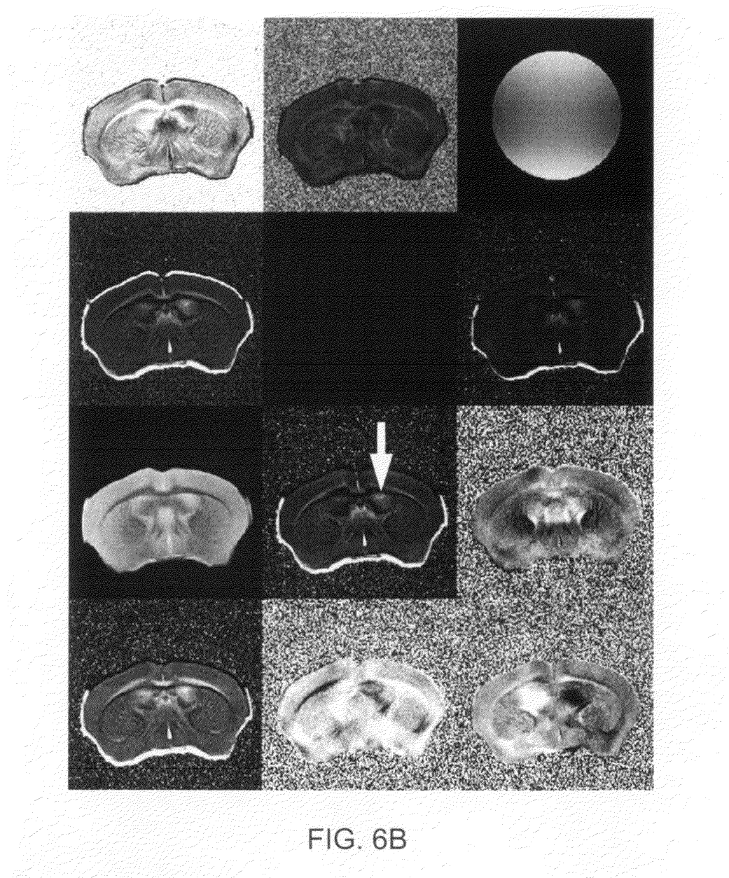

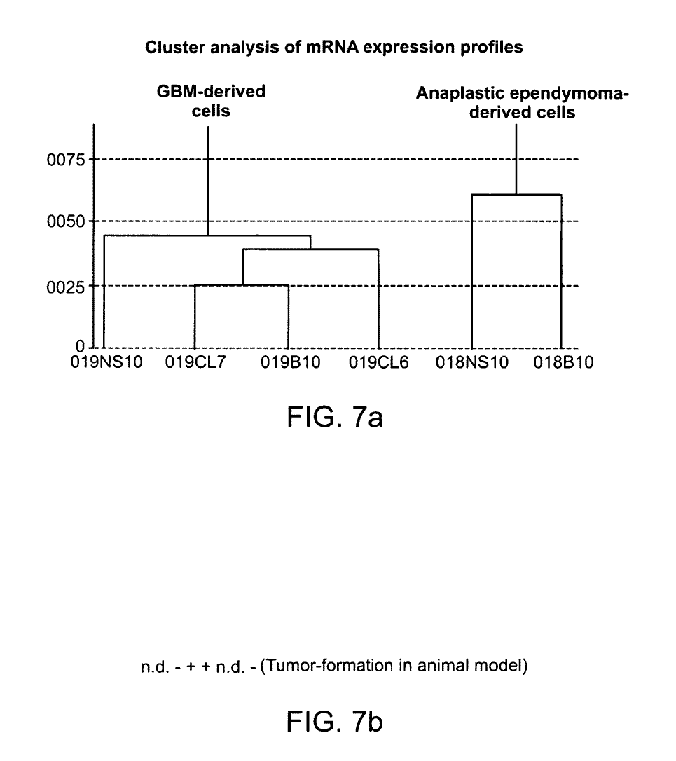

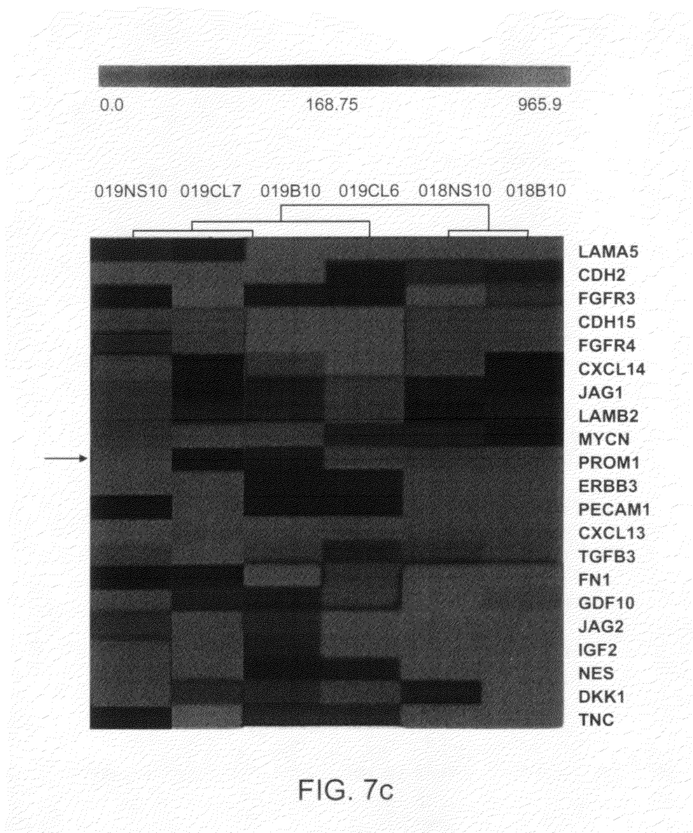

Referring to FIG. 6, T₂-weighted coronal MRI sections demonstrated traits similar to human GBM, including a massive process with typical T₂ heterogeneity in the left hemisphere, a resulting midline shift, and also crossing of the midline with spread far distance of the rostro-caudal axis of the host brain.

### REFERENCES

It is believed that a review of the references will increase appreciation of the present invention. The entire disclosures of all references cited herein are hereby incorporated by reference. All publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.
Cheng L C, Tavazoie M, Doetsch F (2005) Stem cells: from epigenetics to microRNAs. Neuron 46:363-367.
Goetz A K, Scheffler B, Chen H X, Wang S, Suslov O, Xiang H, Brustle O, Roper S N, Steindler D A (2006) Temporally restricted substrate interactions direct fate and specification of neural precursors derived from embryonic stem cells. Proc Natl Acad Sci USA 103:11063-11068.
Hammond S M (2006) MicroRNAs as oncogenes. Curr Opin Genet Dev 16:4-9.
Hayflick L (1973) Tissue culture methods and applications. eds. Kruse P F, Jr. and MK Patterson, Jr. New York, Academic Press: 220.
Henson J W (2006) Treatment of glioblastoma multiforme: a new standard. Arch Neurol 63:337-341.
Kukekov V Q, Laywell E D, Suslov O, Davies K, Scheffler B, Thomas LB, O'Brien T F, Kusakabe M, Steindler D A (1999) Multipotent stem/progenitor cells with similar properties arise from two neurogenic regions of adult human brain. Exp Neurol 156:333-344.
Lee J, Kotliarova S, Kotliarov Y, Li A, Su Q, Donin N M, Pastorino S, Purow B W, Christopher N, Zhang W, Park J K, Fine H A (2006) Tumor stem cells derived from glioblastomas cultured in bFGF and EGF more closely mirror the phenotype and genotype of primary tumors than do serum-cultured cell lines. Cancer Cell 9:391-403.
Massimino M, Biassoni V (2006) Use of high-dose chemotherapy in front-line therapy of childhood malignant glioma. Expert Rev Anticancer Ther 6:709-717.
Masters J R (2000) Human cancer cell lines: fact and fantasy. Nat Rev Mol Cell Biol 1:233-236.
Merchant T E, Fouladi M (2005) Ependymoma: new therapeutic approaches including radiation and chemotherapy. J Neurooncol 75:287-299.
Polyak K, Hahn W C (2006) Roots and stems: stem cells in cancer. Nat Med 12:296-300.
Reynolds B A, Weiss S (1992) Generation of neurons and astrocytes from isolated cells of the adult mammalian central nervous system. Science 255:1707-1710.
Reynolds B A, Rietze R L (2005) Neural stem cells and neurospheres-re-evaluating the relationship. Nat Methods 2:333-336.
Sanai N, Alvarez-Buylla A, Berger M S (2005) Neural stem cells and the origin of gliomas. N Engl J Med 353:811-822.
Scheffler B, Walton N M, Lin D D, Goetz A K, Enikolopov G. Roper S N, Steindler DA (2005) Phenotypic and functional characterization of adult brain neuropoiesis. Proc Natl Acad Sci USA 102:9353-9358.
Segal E, Friedman N, Kaminski N, Regev A, Koller D (2005) From signatures to models: understanding cancer using microarrays. Nat Genet 37 Suppl:S38-45.
Taylor M D, Poppleton H, Fuller C, Su X, Liu Y, Jensen P. Magdaleno S, Dalton J, Calabrese C, Board J, Macdonald T, Rutka J, Guha A, Gajjar A, Curran T, Gilbertson R J (2005) Radial glia cells are candidate stem cells of ependymoma. Cancer Cell 8:323-335.

The invention has been described in detail with reference to preferred embodiments thereof. However, it will be appreciated that those skilled in the art, upon consideration of this disclosure, may make modifications and improvements within the spirit and scope of the invention. It is understood that this invention is not limited to the particular materials and methods described herein. It is also to be understood that the terminology used herein is for the purpose of describing particular embodiments and is not intended to limit the scope of the present invention, which will be limited only by the appended claims.

What is claimed is:

1. A method of isolating a cell population enriched in tumorigenic stem cells, the method comprising:
(a) mincing a tissue sample of a tumor into tissue explants;
(b) plating the tissue explants on a substrate coated with a cell-adhesive layer under conditions that promote attachment of the tissue explants and migration of a subpopulation of cells out of the tissue explants onto the adhesive substrate; and
(c) separating the tissue explants from the migrated cells and dissociating the tissue explants into a single cell suspension, to provide a dissociated cell population and a migratory cell population.

2. The method of claim 1 further comprising:
(d) culturing at least one of said cell populations under conditions that promote proliferation of substantially purified tumorigenic stem cells.

3. An isolated cancer stem cell isolated according to the method of claim 1.

4. An isolated cell population substantially enriched in tumorigenic stem cells derived from a central nervous system (CNS) tumor.

5. An isolated cell population substantially enriched in non-tumorigenic stem cells derived from a central nervous system (CNS) tumor.

6. An isolated cell population substantially enriched in tumorigenic cells that do not possess stem cell characteristics derived from a central nervous system (CNS) tumor.

7. An isolated clonal cell population of tumorigenic stem cells derived from a central nervous system (CNS) tumor.

8. An isolated clonal cell population of non-tumorigenic stem cells derived from a central nervous system (CNS) tumor.

9. An isolated clonal cell population of tumorigenic cells that do not possess stem cell characteristics derived from a central nervous system (CNS) tumor.

10. A method of treatment of a subject with a tumor comprising:
(a) obtaining a tissue sample of the tumor from the subject;
(b) culturing at least one cell population substantially enriched in tumorigenic stem cells derived from the subject's tumor;
(c) identifying an effective therapeutic agent or method to kill or delay the growth of the subject's tumorigenic stem cells; and
(d) administering the effective therapeutic method or agent to the subject to prevent or delay the growth of the tumor.

11. The method of claim 10, wherein the tumor is a central nervous system tumor.

12. A method of classifying a tumor comprising cancer stem cells comprising:
(a) obtaining a tissue sample of a tumor from a subject;
(b) culturing at least one cell population substantially enriched in cancer stem cells derived from the subject's tumor;
(c) identifying one or more biological markers in the cancer stem cells that are expressed at different levels in the stem cells as compared to non-tumorigenic cells of the tumor; and
(d) classifying the tumor on the basis of the presence, or relative proportion, of the biological markers of stem cells as compared with the presence or proportion of said biological markers in other tumors, and in normal control tissues.

13. The method of claim 12, wherein the tumor is a central nervous system (CNS).

14. A method of identifying tumorigenic stem cell markers:
(a) mincing a tissue sample of a tumor into tissue explants;
(b) plating the tissue explants on a substrate coated with a cell-adhesive layer under conditions that promote attachment of the tissue explants and migration of a subpopulation of cells out of the tissue explants onto the adhesive substrate;
(c) isolating migratory cancer stem cells;
(e) implanting the isolated stem cells in an animal model of tumor formation;
(d) characterizing the markers expressed by the tumorigenic and non-tumorigenic stem cells;
thereby determining tumorigenic stem cell markers.

15. The method of claim 14, wherein the marker is not expressed in a non-tumorigenic stem cell.

16. The method of claim 14, wherein the marker is more highly expressed in a tumorigenic stem cell.

17. A method of treating a subject having or at risk of developing cancer comprising; administering to a subject an agent that specifically targets the marker identified in claim 10.

18. The method of claim 17, wherein the agent is an antibody or small molecule.

19. A method of determining if a cancer stem cell is tumorigenic comprising: measuring the amount of prominin 1 (CD133) produced by the cancer stem cell, wherein the absence of expression of prominin 1 (CD 133) is indicative that the cancer stem cell is tumorigenic.
